(19) United States
(12) Patent Application Publication
(10) Pub. No.: US 2012/0029613 A1
(43) Pub. Date: Feb. 2, 2012

(54) Bioerodible Endoprosthesis

(75) Inventors: Liliana Atanasoska, Edina, MN (US); Pankaj Gupta, Minnetonka, MN (US); Dennis A. Boismier, Shorewood, MN (US); Robert W. Warner, Woodbury, MN (US)

(73) Assignee: BOSTON SCIENTIFIC SCIMED, INC., Maple Grove, MN (US)

(21) Appl. No.: 13/156,980

(22) Filed: Jun. 9, 2011

Related U.S. Application Data
(60) Provisional application No. 61/367,929, filed on Jul. 27, 2010.

(51) Int. Cl. A61F 2/82

(52) U.S. Cl. 623/1.15

(57) ABSTRACT

An endoprosthesis includes a composite. The composite includes a matrix comprising a bioerodible iron or a bioerodible iron alloy and particles within the matrix. The particles include palladium, manganese oxide, a transition metal oxide, or a combination thereof.

### CROSS-REFERENCE TO RELATED APPLICATION

This application claims priority under 35 USC §119(e) to U.S. Provisional Patent Application Serial No. 61/367,929, filed on Jul. 27, 2010, the entire contents of which are hereby incorporated by reference.

### TECHNICAL FIELD

This disclosure relates to bioerodible endoprostheses.

### BACKGROUND

The body includes various passageways such as arteries, other blood vessels, and other body lumens. These passageways sometimes become occluded or weakened. For example, the passageways can be occluded by a tumor, restricted by plaque, or weakened by an aneurysm. When this occurs, the passageway can be reopened or reinforced, or even replaced, with a medical endoprosthesis. An endoprosthesis is typically a tubular member that is placed in a lumen in the body. Examples of endoprostheses include stents, covered stents, and stent-grafts.

Endoprostheses can be delivered inside the body by a catheter that supports the endoprosthesis in a compacted or reduced-size form as the endoprosthesis is transported to a desired site. Upon reaching the site, the endoprosthesis is expanded, for example, so that it can contact the walls of the lumen.

The expansion mechanism can include forcing the endoprosthesis to expand radially. For example, the expansion mechanism can include the catheter carrying a balloon, which carries a balloon-expandable endoprosthesis. The balloon can be inflated to deform and to fix the expanded endoprosthesis at a predetermined position in contact with the lumen wall. The balloon can then be deflated, and the catheter withdrawn.

In another delivery technique, the endoprosthesis is formed of an elastic material that can be reversibly compacted and expanded, e.g., elastically or through a material phase transition. During introduction into the body, the endoprosthesis is restrained in a compacted condition. Upon reaching the desired implantation site, the restraint is removed, for example, by retracting a restraining device such as an outer sheath, enabling the endoprosthesis to self-expand by its own internal elastic restoring force.

It is sometimes desirable for an implanted endoprosthesis to erode over time within the passageway. For example, a fully erodible endoprosthesis does not remain as a permanent object in the body, which may help the passageway recover to its natural condition. Bioerodible endoprostheses can be formed from, e.g., a polymeric material, such as polylactic acid, or from a metallic material, such as magnesium, iron or an alloy thereof.

Bioerodible metals can erode due to corrosion in vivo. The corrosion process, however, can be non-uniform due to localized attacks and difficult to control. In vivo corrosion rates are difficult to predict from in vitro data. Accordingly, it is difficult to design a bioerodible endoprosthesis having the desired structural integrity for a desired period of time.

### SUMMARY

An endoprosthesis is disclosed that includes a composite including a matrix of a bioerodible iron or bioerodible iron alloy and a plurality of particles within the matrix. The particles include palladium, manganese oxide, one or more transition metal oxides, or a combination thereof.

The particles can have diameters of less than 50 micrometers. In some embodiments, the particles have diameters of between 0.5 micrometers and 10 micrometers. In other embodiments, the particles are nanoparticles having diameters of between 20 nm and 500 nm.

The particles can include a core and a shell. In some embodiments, the core can include iron, magnesium, cobalt, zinc, copper, or a combination thereof and the shell includes palladium. In other embodiments, the core comprises magnesium and the shell includes manganese oxide.

In some embodiments including palladium, the palladium can be at least 99 percent pure. In some embodiments, the composite includes between 0.5 and 5 weight percent of palladium.

In some embodiments including manganese oxide, the manganese oxide is selected from the group of MnO₂, Mn₃O₄, and combinations thereof. In some embodiments, the manganese oxide is mixed with a transition metal oxide.

The bioerodible iron alloy can be an alloy including iron and manganese. In some embodiments, the alloy includes at least 90 weight percent iron and less than 10 weight percent manganese. In some embodiments, the alloy includes less than 5 weight percent manganese.

The endoprosthesis can be a stent.

The particles can act as an oxidation reduction catalyst that accelerates the corrosion rate of the bioerodible iron or bioerodible iron alloy when the endoprosthesis is within a physiological environment,

The details of one or more embodiments are set forth in the accompanying drawings and the description below. Other features, objects, and advantages will be apparent from the description and drawings, and from the claims.

### DESCRIPTION OF DRAWINGS

FIG. 1 is an example of a stent.

FIG. 2 depicts a cross-section of a stent strut body including a matrix of bioerodible iron or a bioerodible iron alloy and a plurality of particles within the matrix.

FIG. 3 depicts the structure of a nanoparticle.

FIG. 4 is a perspective view of an artificial heart valve in an expanded configuration.

Like reference symbols in the various drawings indicate like elements.

### DETAILED DESCRIPTION

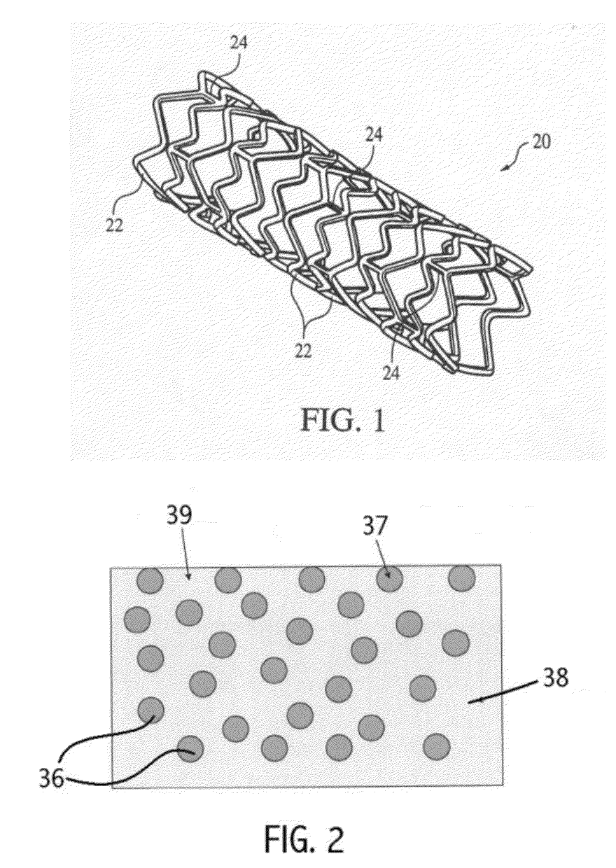

Stent 20, shown in FIG. 1, is discussed below as an example of one endoprosthesis according to the instant disclosure. Stent 20 includes a pattern of interconnected struts forming a structure that contacts a body lumen wall to maintain the patency of the body lumen. For example, stent 20 can have the form of a tubular member defined by a plurality of bands 22 and a plurality of connectors 24 that extend between and connect adjacent bands. During use, bands 22 can be expanded from an initial, small diameter to a larger diameter to contact stent 20 against a wall of a vessel, thereby maintaining the patency of the vessel. Connectors 24 can provide stent 20 with flexibility and conformability that allow the stent to adapt to the contours of the vessel. Other examples of endoprostheses can include covered stents, stent-grafts, and artificial heart valves.

Stent 20 is a composite of a matrix of a bioerodible iron or a bioerodible iron alloy and a plurality of particles with the matrix. The term “composite,” as used herein, requires the presence of two or more constituent materials that remain separate and distinct within the finished structure. A “composite” is not an alloy, i.e., a solid solution. Instead, the particles remain compositionally distinct from the bioerodible iron or bioerodible iron alloy of the matrix. The particles are not precipitates within a bioerodible iron alloy.

The particles include an oxidation reduction catalyst that increases the rate of corrosion of the matrix within a physiological environment. Under physiological conditions, the corrosion reaction of iron is cathodically controlled and the corrosion current is at least partially determined by the limiting diffusion current for the oxygen reduction reaction. The corrosion reaction is more likely to be limited by the diffusion current for the oxygen reduction reaction when the iron is within an acidic environment. The addition of an oxidation reduction catalyst to stent 20 as part of a composite structure can accelerate the corrosion of the iron and can ensure that the stent struts degrade in a controlled manner.

FIG. 2 depicts a cross-section of a stent strut (band 22 or connector 24). Exposed portions of the discrete particles 36 act as oxidation reduction catalyst sites 37 and adjacent areas of the bioerodible iron or bioerodible iron alloy act as anodic sites 39. As the bioerodible iron or bioerodible iron alloy erodes within the physiological environment, discrete particles 36 are released and new particles 36 become exposed to the physiological environment. In some embodiments, endothelialization of the stent 20 can prevent the particles 36 from being released into the blood stream. The particles 36 can be sized such that the release of the particles into the blood stream does not result in embolisms. In some embodiments, the particles 36 can have a maximum diameter of 50 micrometers. For example, the particles 36 can have diameters of between 0.5 and 10 micrometers. In some embodiments, the particles are nanoparticles having a diameter of between 20 nm and 500 nm. The concentration and distribution of the particles 36 within the matrix 38 can be varied to impact the erosion rates of the iron in different portions of the stent 20. As will be discussed below, the composite of an oxidation reduction catalysts and a matrix of bioerodible iron or a bioerodible iron alloy can be formed using a powder metal sintering process, which can be used to prevent the particles 36 from being alloyed with the iron or iron alloy.

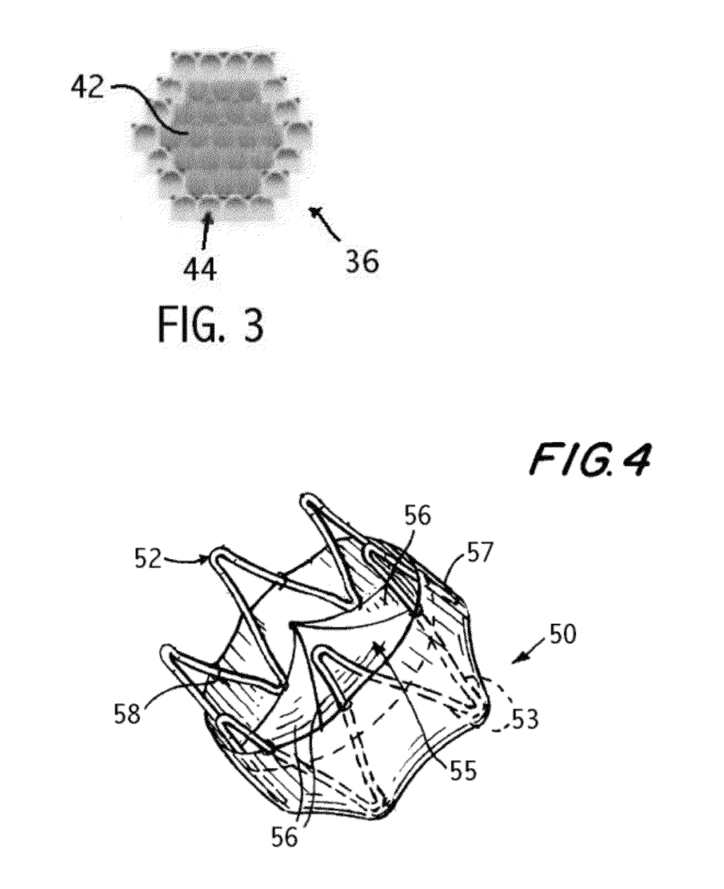

The oxidation reduction catalyst is palladium in some embodiments. The palladium can be at least 95 percent by weight pure. In some embodiments, the palladium is at least 99 percent by weight pure. In some embodiments, the nanoparticles can consist solely of palladium. In other embodiments, the nanoparticles can have a shell of palladium over a core. The core can include iron, magnesium, cobalt, zinc, copper, or a combination thereof. FIG. 3 depicts an example of a nanoparticle 36 having a core 42 and a shell of palladium 44. The palladium 44 may be a single atomic layer or include multiple layers. In some embodiments, the nanoparticles 36 can include additional intermediate layers. For example, nanoparticles having a shell of palladium can be formed by forming an alloy of palladium with iron, cobalt, zinc, or copper and shaping the alloy into nanoparticles. The constituents of the alloy can then be segregated such that the palladium moves to the surface of the nanoparticle by elevating the temperature of the nanoparticles. An example of a similar process is described in K. Gong et al., J. Electroanal. Chem. (2010), doi:10.1016/j.jelechem.2010.04.011. Additional layers of palladium can be deposited by depositing molecular layers of copper using under potential deposition (“UPD”) and replacing the copper with palladium.

The oxidation reduction catalyst is manganese oxide in some embodiments. In some embodiments, the manganese oxide is Mn₃O₄, MnO₂, or a combination thereof. Manganese metal and MnO do not have the same catalytic effect as Mn₃O₄ or MnO₂ because the crystal structure of the manganese oxide affects the catalytic performance. The manganese oxide can overlie a body of bioerodible iron or a bioerodible iron alloy and/or can be in the form of particles within a matrix of a bioerodible iron or bioerodible iron alloy. In some embodiments, a matrix of a bioerodible iron or a bioerodible iron alloy can include nanoparticles of manganese oxide. The nanoparticles can have an average diameter of between 20 nm and 500 nm. In some embodiments, the manganese oxide is in the form of nanoparticles having a shell of manganese oxide overlying a core. The core, in some embodiments, can be a bioerodible metal that breaks down in the physiological environment to produce basic byproducts. In some embodiments, the core is magnesium. A shell of manganese oxide over a magnesium core can prevent the magnesium from galvanically polarizing the iron.

The oxidation reduction catalyst can include a transition metal oxide, such as ruthenium dioxide. For example, manganese oxide can be mixed with a transition metal oxide. In some embodiments, stent 20 include a combination of different types of oxidation reduction catalysts. For example, a stent 20 can include multiple particles of palladium, manganese oxide, and ruthenium dioxide within a matrix of bioerodible iron or a bioerodible iron alloy.

Stent 20 includes a matrix featuring a bioerodible iron or bioerodible iron alloy. In some embodiments, the matrix includes substantially pure iron (e.g., greater than 99% pure iron). Bioerodible iron alloys can include at least 65% by weight iron. For example, the bioerodible metal portion can include a bioerodible iron alloy that includes up to twenty percent manganese, up to 10 percent silver, and up to five percent carbon. For example, in some embodiments, a bioerodible iron alloy can include at least 90 weight percent iron and less than 10 weight percent manganese. In some embodiments, the alloy includes less than 5 weight percent manganese. In some embodiments, the alloy includes at least 1 weight percent manganese. For example, a stent can include a matrix of an iron-manganese alloy having 95-99 weight percent iron and 1-5 weight percent manganese and a plurality of nanoparticles of palladium within the matrix, the composite comprising a total of 0.5 to 5 weight percent palladium.

The composite can be produced using conventional techniques. In some embodiments, the composite is formed to include between 0.1 and 30 percent by weight of the oxidation reduction catalyst. In some embodiments, the composite includes between 0.5 and 20 weight percent of the oxidation reduction catalyst. The composite can include less than 10 weight percent of the oxidation reduction catalyst. For example, a composite of palladium and a bioerodible iron or bioerodible iron alloy can include between 0.5 and 5 weight percent palladium. A composite of manganese oxide and a bioerodible iron or bioerodible iron alloy can include between 0.5 and 5 weight percent manganese oxide.

The composite can be formed by powder sintering methods. Particles of the oxidation reduction catalyst can be mixed with powder of the bioerodible iron or bioerodible iron alloy. The mixture of powders can then be pressed and heated to a temperature below the melting point of the oxidation reduction catalyst, but sufficient to cause the bioerodible iron or bioerodible iron alloy particles to adhere. Because the powders are not heated above the melting point of the oxidation reduction catalyst, the oxidation reduction catalyst does not alloy with the iron. A sintered bar or tube can then be further worked and shaped into the desired dimensions of a stent by press rolling and other mechanical shaping techniques. In some embodiments, the oxidation reduction catalysts are in the form of nanoparticles having a core and shell structure, with the shell containing the oxidation reduction catalyst.

Stent 20 can be configured for vascular, e.g., coronary and peripheral vasculature or non-vascular lumens. For example, they can be configured for use in the esophagus or the prostate. Other lumens include biliary lumens, hepatic lumens, pancreatic lumens, urethral lumens.

Stent 20 can be of a desired shape and size (e.g., coronary stents, aortic stents, peripheral vascular stents, gastrointestinal stents, urology stents, tracheal/bronchial stents, and neurology stents). Depending on the application, the stent can have a diameter of between, e.g., about 1 mm to about 46 mm. In certain embodiments, a coronary stent can have an expanded diameter of from about 2 mm to about 6 mm. In some embodiments, a peripheral stent can have an expanded diameter of from about 4 mm to about 24 mm. In certain embodiments, a gastrointestinal and/or urology stent can have an expanded diameter of from about 6 mm to about 30 mm. In some embodiments, a neurology stent can have an expanded diameter of from about 1 mm to about 12 mm. An abdominal aortic aneurysm (AAA) stent and a thoracic aortic aneurysm (TAA) stent can have a diameter from about 20 mm to about 46 mm. The stent can be balloon-expandable, self-expandable, or a combination of both (e.g., see U.S. Pat. No. 6,290,721).

Stent 20 can also be part of a covered stent, a stent-graft and/or other endoprostheses. The endoprosthesis, in some embodiments, can an artificial heart valve. For example, an artificial heart valve 50 is depicted in FIG. 4. The heart valve 50 has a generally circular shape. A stent member 52 is formed of a wire including a composite of bioerodible iron or a bioerodible iron alloy with an oxidation reduction catalyst. The stent member 52 is formed in a closed zig-zag configuration. In other embodiments, the stent member of the artificial heart valve can include a plurality of bands with connectors in between. The valve member 55 is flexible and includes a plurality of leaflets 56. The leaflet portion of the valve member 55 extends across or transverse of the cylindrical stent member 52. The leaflets 56 are the actual valve and allow for one-way flow of blood. Extending from the periphery of the leaflet portion is a cuff portion 57. The cuff portion is attached to the stent by sutures 58. Sutures 53 can be used to attach the artificial heart valve 50 to heart tissue. The valve member 55 can be formed of polymer such as polytetrafluoroethylene or a polyester. In other embodiments, the valve member 55 can be a bioerodible polymer.

All publications, patent applications, patents, and other references mentioned herein are incorporated by reference herein in their entirety.

A number of embodiments have been described. Nevertheless, it will be understood that various modifications may be made without departing from the spirit and scope of this disclosure. Accordingly, other embodiments are within the scope of the following claims.

What is claimed is:

1. An endoprosthesis comprising:
a composite comprising:
a matrix of a bioerodible iron or bioerodible iron alloy; and
a plurality of particles within the matrix, the particles comprising palladium.

2. The endoprosthesis of claim 1, wherein the particles have diameters of between 0.5 micrometers and 10 micrometers.

3. The endoprosthesis of claim 1, wherein the palladium is at least 99 percent pure.

4. The endoprosthesis of claim 1, wherein the particles are nanoparticles having diameters of between 20 nm and 500 nm.

5. The endoprosthesis of claim 1, wherein the particles comprise a core and a shell, wherein the core comprises iron, magnesium, cobalt, zinc, copper, or a combination thereof and the shell comprises palladium.

6. The endoprosthesis of claim 1, wherein the bioerodible iron or bioerodible iron alloy is an alloy comprising iron and manganese.

7. The endoprosthesis of claim 6, wherein the alloy comprises at least 90 weight percent iron and less than 10 weight percent manganese.

8. The endoprosthesis of claim 7, wherein the alloy comprises less than 5 weight percent manganese.

9. The endoprosthesis of claim 1, wherein the composite comprises between 0.5 and 5 weight percent palladium.

10. The endoprosthesis of claim 1, wherein the endoprosthesis is a stent.

11. An endoprosthesis comprising:
a composite comprising:
a matrix of a bioerodible iron or bioerodible iron alloy; and
a plurality of particles within the matrix, the particles comprising manganese oxide.

12. The endoprosthesis of claim 11, wherein the particles have diameters of between 0.5 micrometers and 10 micrometers.

13. The endoprosthesis of claim 11, wherein the particles are nanoparticles having diameters of between 20 nm and 500 nm.

14. The endoprosthesis of claim 11, wherein the plurality of particles comprise a core of magnesium having an outer surface comprising manganese oxide.

15. The endoprosthesis of claim 11, wherein the manganese oxide is selected from the group consisting of MnO₂, Mn₃O₄, and combinations thereof

16. The endoprosthesis of claim 11, wherein the manganese oxide is mixed with a transition metal oxide.

17. The endoprosthesis of claim 11, wherein the endoprosthesis is a stent.

18. An endoprosthesis comprising:
a composite comprising:
a matrix comprising a bioerodible iron or a bioerodible iron alloy; and
nanoparticles within the matrix, the nanoparticles comprising an oxidation reduction catalyst that accelerates the corrosion rate of the bioerodible iron or bioerodible iron alloy when the endoprosthesis is within a physiological environment, wherein the oxidation reduction catalyst is selected from the group consisting of palladium, manganese oxide, transition metal oxides, and combinations thereof

19. The endoprosthesis of claim 18, wherein the composite comprises between 0.5 and 5 weight percent of the oxidation reduction catalyst.

20. The endoprosthesis of claim 18, wherein the endoprosthesis is a stent.
